(19) United States
(12) Patent Application Publication
(10) Pub. No.: US 2011/0150964 A1
(43) Pub. Date: Jun. 23, 2011

(54) APTAMER-COATED IMPLANT, PROCESS OF PRODUCTION, AND USES

(76) Inventor: Alexander Borck, Aurachtal (DE)

(21) Appl. No.: 12/963,949

(22) Filed: Dec. 9, 2010

Related U.S. Application Data
(60) Provisional application No. 61/288,361, filed on Dec. 21, 2009.

(51) Int. Cl. A61F 2/00; A61K 31/7088

(52) U.S. Cl. 424/423; 514/44 R

(57) ABSTRACT

One embodiment of the present invention is an implant with an aptamer coating, wherein the aptamer has an activating effect on the αᵥβ₃ integrin receptor, for improving the endothelial cell adhesion to the implant, a process for producing same, and uses thereof.

### CROSS REFERENCE

The present application claims priority on U.S. Provisional Application No. 61/288,361, filed on Dec. 21, 2009, which application is incorporated by reference herein.

### TECHNICAL FIELD

A field of the invention is medical implants. Another field is stents. One embodiment of the present invention relates to an aptamer-coated implant, wherein the aptamer has an activating effect on the αᵥβ₃ integrin receptor, for improving the endothelial cell adhesion to the implant, a process for producing same, and uses thereof.

### BACKGROUND

Implants are substances or parts which are introduced into the human or animal body for performing certain replacement functions for a limited time period or for a lifetime. In contrast to transplants, implants are composed of synthetic material (alloplastic). Distinctions are frequently made among medical, plastic, or functional implants.

The purpose of medical implants is to support or replace bodily functions. Depending on the function, they are also referred to as implantable prostheses. Examples of known representatives include (electrodes for) cardiac pacemakers, cardiac implants such as cardiac values and shunts, (coronary and peripheral) stents, and neurostents.

In general, stents are endovascular (peripheral or coronary) prostheses or implants, known, for example, for treatment of stenoses and also aneurysms. Stents basically comprise a support structure which is suitable for appropriately supporting the wall of a vessel so as to expand the vessel or bridge an aneurysm. For this purpose, stents are inserted into the blood vessel in a compressed state, and are then expanded at the treatment site and pressed against the vascular wall. This expansion may be achieved using an angioplasty balloon, for example, which is mounted on an insertable catheter. Alternatively, self-expanding stents are also known. Such stents are composed, for example, of a superelastic metal such as Nitinol.

Stents are currently divided into two basic types: permanent stents and degradable stents. Permanent stents are designed to remain in the blood vessel for an indefinite period of time. In contrast, degradable stents are degraded in a vessel over a predetermined period of time.

For cardiovascular diseases, minimally invasive treatment forms such as the expansion and stabilization of stenotic coronary vessels using percutaneous transluminal coronarangioplasty (PTCA) in combination with stent implantation represents an increasingly common treatment method. As a late complication, besides restenosis of the vessel after PTCA or stent implantation (in-stent restenosis (ISR)) and tissue inflammation in the vicinity of the PTCA treatment and/or the stent implantation, above all there is the risk of a thromboembolism. For this reason, in particular stents in the prior art are provided which are coated with active substances (so-called “drug-eluting stents” (DES)) with the aim of counteracting one or more late complications. However, DES which act against intimal hyperplasia, for example, have the adverse side effects of necrosis of the vascular tissue in the vicinity of the stent implantation on the one hand, and long-term damage such as late thrombosis on the other hand.

The physical anchoring of cells is performed primarily by integrins, which as heterodimeric glycoproteins are also used for bidirectional signal transduction through the cellular wall. Binding of a ligand to the integrin causes a signal to be transmitted into the cell, resulting in reorientation of the cytoskeleton. The process referred to as “outside-in signaling” may control modification of the cell form, migration, or anchoring-dependent proliferation, as well as tissue organization. The αᵥβ₃ integrin receptor has many different ligands, and is found in particular on endothelial cells, mature, bone-resorbing osteoclasts, migrating smooth muscle cells, tumor cells, platelets, fibroblatsts, activated macrophages/monocytes, and neutrophilic granulocytes. Accordingly, the αᵥβ₃ integrin receptor plays a particularly important role in atherogenesis, bone resorption, neovascularization/angiogenesis, and inflammatory events.

One purpose of the present invention is to provide an implant with increased biocompatibility, and preferably to increase the healing of the implant in the tissue and/or to reduce the risk of late complications.

### SUMMARY

A first embodiment of the invention concerns an implant for a human or animal body, characterized in that an implant base body includes a complete or partial coating containing one or more aptamers which are the same or different, wherein the aptamer(s) have an activating effect on the αᵥβ₃ integrin receptor.

A second embodiment of the invention concerns a process for producing an implant according to the invention, characterized in that the process comprises or is composed of the following steps:
a) Providing an implant base body,
b) Providing a coating material comprising one or more aptamers which are the same or different and which have an activating effect on the αᵥβ₃ integrin receptor, the coating material being suitable for releasing the aptamer(s) from the coating under physiological conditions, and
c) Applying the coating material from step b) to at least a portion of the surface and/or in a cavity or cavities of the implant base body.

A third embodiment of the invention concerns a process for producing an implant wherein it is essentially not possible for the aptamer(s) to be released from the coating under physiological conditions, characterized in that the process comprises or is composed of the following steps:
a) Providing an implant base body,
b) Providing one or more anchor groups which are the same or different,
c) Functionalizing the implant base body from step a) using the anchor group(s) from step b),
d) Providing one or more aptamers which are the same or different and which have an activating effect on the αᵥβ₃ integrin receptor, and
e) Binding the aptamer(s) from step d) to the functionalized implant base body or bodies from step c).

A fourth embodiment of the invention concerns use of an aptamer having an activating effect on the αᵥβ₃ integrin receptor for mediation of endothelial cell adhesion to an implant for a human or animal body.

A fifth embodiment of the invention concerns use of one or more different aptamers having an activating effect on the αᵥβ₃ integrin receptor for the complete or partial coating of an implant base body for a human or animal body.

A sixth embodiment of the invention concerns use of one or more different aptamers having an activating effect on the αᵥβ₃ integrin receptor for producing an implant.

Embodiments of the invention are explained herein, including others than those summarized above, and elements thereof may be combined with one another if expedient for one skilled in the art.

### DETAILED DESCRIPTION

At least some aspects of embodiments of the present invention are based on the discovery that due to their activating effect on the αᵥβ₃ integrin receptor, aptamers to be used according to invention embodiments specifically mediate endothelial cell adhesion to the implant surface, and, therefore, accompanying formation of an endothelial layer which is comparable to healthy tissue, thereby increasing the biocompatibility of implants. In addition to the accelerated healing of the implant, an accelerated overgrowth of the implant with endothelial cells and formation of a functional endothelial layer, i.e., corresponding to healthy tissue, may also reduce inadequate proliferation of smooth muscle cells mediated by the implantation and/or excessive formation and secretion of components of the extracellular matrix. In the case of coronary stents as preferred implants according to the invention, this means that the risk of restenosis and/or late thrombosis may be reduced. These and other important advantages are achieved through invention embodiments.

Aptamers to be used according to some invention embodiments have the advantage that, in particular due to their three-dimensional structure, on the one hand they are able to bind to the αᵥβ₃ integrin receptor more selectively and/or more intensely than the typically used (c)RGD peptides, and thus reduce the risk of nonspecific integrin activations. On the other hand, aptamers to be used according to some invention embodiments are also more stable compared to (c)RGD peptides with respect to sterilization treatment during implant manufacture as well as physiological degradation processes. The use of aptamers according to embodiments of the invention, in comparison to use of released active substances from the prior art, are believed to result in advantages including reduced adverse side effects, such as inflammation of tissue in the vicinity of the stent implantation or late thrombosis.

Within the meaning of the present invention, “aptamer with an activating effect on the αᵥβ₃ integrin receptor” means that single-strand oligo- or polynucleotides, further single-strand ribonucleic acid (RNA), deoxyribonucleic acid (DNA), and/or chemically modified nucleic acids, are used which due to their three-dimensional structure bind to the αᵥβ₃ integrin receptor on endothelial cells in such a way that the αᵥβ₃ integrin receptor is transformed from the inactive to the active conformation and triggers the corresponding signal transduction (so-called “outside-in-signaling”), which results in endothelial cell adhesion to the implant.

Suitable aptamers according to invention embodiments may be prepared using customary methods, in particular the so-called systematic evolution of ligands by exponential enrichment (SELEX) process. The SELEX process is based on two different methods for in vitro selection of nucleic acids, published in 1990 by Tuerk & Gold (Tuerk, C., Gold, L. (1990), “Systematic evolution of ligands by exponential enrichment: RNA ligands to bacteriophage T4 DNA polymerase,” Science 249(4968):505-510), and Ellington & Szostak (Ellington, A. D., Szostak, J. W. (1990), “In vitro selection of RNA molecules that bind specific ligands,” Nature 346(6287):818-822).

The SELEX process of Tuerk & Gold begins with the production of 10¹⁴-10¹⁵ different randomized RNA sequences which fold in different ways due to the variation in their primary sequences. This library is obtained by slightly modifying a region of a structurally known RNA oligonucleotide. The primer sequences at the 3′ and 5′ ends remain the same. This library is then incubated with the target protein, and the bound RNAs are subsequently amplified using the reverse transcriptase polymerase chain reaction (RT-PCR). By means of in vitro transcription a new library is then formed which is enriched with RNA which binds to the target protein. This selection and amplification process is then repeated at least four times until the RNA ligands with the greatest affinity for the target protein have been isolated. The ligands thus obtained are then cloned and sequenced. The method is accordingly based on the processes of evolution, variation, selection, and replication.

The method of Ellington & Szostak describes the selection of RNA oligonucleotides via columns, using affinity chromatography. The RNA library that was produced in that article consisted of 10¹³ different RNA molecules. The amplification of the obtained RNA ligands was likewise enabled by PCR. After at least five rounds the selected oligonucleotides were cloned and sequenced. Ellington & Szostak were the first to use the term “aptamers.”

These basic SELEX processes have been further optimized, and are known in the art.

Aptamers to be used according to at least some invention embodiments having an activating effect on the αᵥβ₃ integrin receptor may also be prepared, for example, by the fact that they comprise or are composed of oligonucleotides of aptamers which bind to the αᵥβ₃ integrin receptor but inhibit endothelial cell adhesion, and one or more of the phosphate groups of the sugar-phosphate backbone are derivatized to one or more thiophosphate and/or methyl phosphate groups which are the same or different, preferably thiophosphate, dithiophosphate, and methyl phosphate. As an example, formula 1 shows a section of a 5′-3′-thiophosphate bond between two nucleic acids (B stands for nucleic base):

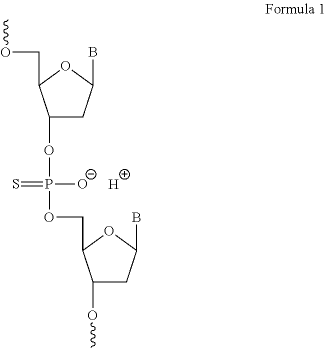

Thiophosphate group derivatizations may be carried out as follows:

A thiophosphate group may be prepared, for example, by the action of elemental sulfur in CS₂ on hydrogen phosphonate, or by sulfonation of phosphite trieseters with tetraethylthiuram disulfide (TETD) or 3H-1,2-bensodithiol-3-one-1,1-dioxide (BDTD) solution (see http://www.sigmaaldrich.com/life-science/custom-oli gos/custom-dna/learning-center/phosphorothioates.html). The latter method avoids the problem of insolubility of the sulfur in most organic solvents and the toxicity of CS₂. The TETD and BDTD methods also typically result in higher-purity thiophosphates.

Although thiophosphate or methyl phosphate derivatizations are known in the art for making nucleic acids more stable with regard to RNase degradation. According to at least some embodiments of the invention, it has surprisingly been discovered that as a result of corresponding derivatizations it is possible not only to obtain nucleic acids which are more stable with regard to RNase splitting, but also that aptamers derivatized according to the invention also have an activating effect on the αᵥβ₃ integrin receptor and thus mediate endothelial cell adhesion.

Aptamers which bind to the αᵥβ₃ integrin receptor but inhibit endothelial cell adhesion are described in the following publications, for example: “Aptamer selection for the inhibition of cell adhesion with fibronectin as target,” Ogawa, A. et al., Bioorganic & Medicinal Chemistry Letters 14 (2004) 4001-4004 (including SEQ ID 1); “Targeted inhibition of alphavbeta3 integrin with an RNA aptamer impairs endothelial cell growth and survival,” Mi, J. et al., Biochem Biophys Res Commun 2005, 338:956-963, which are herein incorporated by reference.

In one embodiment, an aptamer comprises or is composed of at least 50%, further 60 to 100%, of the nucleic acid or nucleotides of an aptamer which binds to the αᵥβ₃ integrin receptor, but inhibits endothelial cell adhesion, whereby preferably between 5 and 80%, still further 10 to 40%, of the phosphate groups are derivatized to thiophosphate and/or methyl phosphate groups. The derivatizations may be situated on the 5′ or 3′ end or may be statistically distributed.

In a further embodiment, an aptamer comprises or is composed of SEQ ID NO:1, whereby between 1 and 80, in other embodiments 3 to 20, in still further embodiments 5 to 12, phosphate groups are derivatized to thiophosphate and/or methyl phosphate groups.

In addition, suitable aptamers may be modified in such a way that they are protected (reduced degradation by nucleases), particularly under physiological conditions, without losing their effectiveness. Examples of corresponding protective methods include the Spiegelmer® technology as well as the locked nucleic acids (LNA) technology. In the Spiegelmer® technology, the beneficial properties of the aptamers may be conveyed to mirror-image nucleic acids which are composed of L-nucleotides and which therefore cannot be recognized by nucleic acid-degrading enzymes, for example ubiquitous RNases. These Spiegelmers® also exhibit high stability in the blood serum. In addition to the Spiegelmers®, however, it is also possible to stabilize selected functional nucleic acids against nuclease degradation by modifying the sugar-phosphate backbone. Methylation and fluorination, for example, may be carried out, or locked nucleic acids (LNA) may be incorporated into the aptamers for stabilization.

Implants or implant base bodies may be any medical, plastic, and/or functional implants or implant base bodies, and are selected, for example, from the group comprising cardiac pacemakers; brain pacemakers; cardiac implants; pacemaker electrodes; defibrillation electrodes; cochlear implants; dental implants; endoprostheses, preferably for knee joints and/or hip joints; depot implants used to form a depot for an active substance; degradable or permanent coronary or peripheral stents; degradable or permanent stents for other cavities, further the esophagus, bile duct, urethra, prostate, or trachea; and local drug delivery (LDD) implants which may be, for example, implanted endovascularly in the blood or other cavities.

Embodiments of the present invention are also particularly well suited for bone implants, since the αᵥβ₃ integrin receptor is also formed on mature bone-resorbing osteoclasts, and thus comprises a substantial fraction for bone resorption.

In one embodiment of the present invention, implants or implant base bodies can be, for example, selected from the group comprising cardiac pacemakers; cardiac implants; pacemaker electrodes; defibrillation electrodes; shunts; degradable or permanent coronary or peripheral stents; and neurostents and local drug delivery (LDD) implants which may be, for example, implanted endovascularly in the blood or other cavities.

In one embodiment, implants or implant base bodies may be, for example, selected from the group comprising permanent or degradable coronary stents, wherein the stent base body material may include, for example, metal and/or polymer.

In one embodiment, the original mechanical functions of a coronary stent, for example, dilation capability, low recoil, stability over a desired period of time (in the case of degradable stents composed, for example, of magnesium and alloys thereof), and flexibility, may be preserved.

Implant (base body) materials typically to be used according to the invention, preferably stent base body materials, and preferred embodiments thereof are described below, with metal being preferred over polymer in at least some embodiments, since in particular degradation products of degradable polymer implants themselves have increased risk for inflammation and thromboses.

### Degradable Implant Base Body:

Within the meaning of the present invention, “degradable implant (base body),” or “degradable stent (base body),” (also used synonymously with “base body” below) means that the base body degrades in the physiological surroundings, for example, in the vascular system of a human or animal organism, i.e., is degraded in such a way that the stent loses its integrity. In further embodiments, the degradable base bodies degrade only when the functions of the implant are no longer physiologically appropriate or necessary. For some degradable stents, this means that the stent may degrade or lose its integrity only when the traumatized tissue of the vessel has healed, and the stent therefore no longer needs to remain in the vessel lumen.

### Metallic Base Body:

In one embodiment of the present invention, the degradable material comprises or is composed of a metallic material or a biocorrodable alloy. In further embodiments, the main component of the alloy being selected from the group comprising magnesium, iron, zinc, and/or tungsten. In still further embodiments, a magnesium alloy may be used for a degradable metallic material.

In some embodiments, an alloy could be selected with respect to its composition so that it is biocorrodable. In further embodiments an alloy selected to be biocorrodable could contain magnesium, iron, zinc and/or tungsten. Within the meaning of the present invention, “biocorrodable” refers to alloys for which degradation takes place in the physiological surroundings, with the ultimate result that the entire stent or the portion of the stent formed from the material loses its mechanical integrity. In some embodiments, “alloy” refers to a metallic structure. In further embodiments, “alloy” refers to a metallic structure whose main component is magnesium, iron, zinc, and/or tungsten. The main component is the component having the highest weight fraction in the alloy. In some embodiments, the fraction of the main component is greater than 50% by weight, in further embodiments greater than 70% by weight. In further embodiments, a magnesium alloy may be used.

In an invention embodiment containing magnesium alloy, said alloy may, for example, contain yttrium and other rare earth metals. Such an alloy is distinguished by its physical-chemical properties, high biocompatibility, and degradation products.

A further embodiment uses magnesium alloys of the WE series, in particular WE43, and magnesium alloys having the composition of 5.5 to 0.9% by weight rare earth metals, of which 0.0 to 5.5% by weight is yttrium and the remainder is <1% by weight, whereby the remainder may contain zirconium and/or silicon, and magnesium may account for the rest up to 100% by weight. These magnesium alloys have been confirmed experimentally and in initial clinical trials; i.e., the alloys exhibit high biocompatibility, favorable processing characteristics, good mechanical parameters, and corrosion behavior which is adequate for the intended purpose. The collective term “rare earth metals” in the present application may refer, for example, to scandium (21), yttrium (39), lanthanum (57), and the fourteen elements following lanthanum (57), namely, cerium (58), neodymium (60), promethium (61), samarium (62), europium (63), gadolinium (64), terbium (65), dysprosium (66), holmium (67), erbium (68), thulium (69), ytterbium (70), and lutetium (71).

Polymer Base Body:

In an embodiment of to the invention, implant base bodies, and further stent base bodies, may comprise or be composed of degradable polymer, including but not limited to: polydioxanone; polyglycolide; polycaprolactone; polyhydroxyvaleric acid; polyhydroxybutyric acid; polylactides, preferably poly(L-lactide), poly(D-lactide), poly(D,L-lactide), and blends thereof, and copolymers, preferably poly(L-lactide-coglycolide), poly(D,L-lactide-coglycolide), poly(L-lactide-co-D,L-lactide), poly(L-lactide-cotrimethylene carbonate), and triblock copolymers; polysaccharides, preferably chitosan, levan, hyaluronic acid, heparin, dextran, and cellulose.

### Permanent Implant Base Body:

In contrast to the degradable base body, a permanent implant base body, and further a permanent stent base body, may be essentially undegraded in the physiological surroundings of a human or animal organism, and thus does not lose its integrity.

In an embodiment of the invention, the base body of a permanent implant, and further of a permanent stent, may be made from a shape memory material, further one or more materials may be selected from the group of nickel-titanium alloys and copper-zinc-aluminum alloys, particularly Nitinol.

In another embodiment of the invention, the base body of a permanent implant, further a permanent stent, is composed of stainless steel, further Cr—Ni—Fe steel, still further the alloy 316L, or a Co—Cr steel.

In a further embodiment of the invention, the implant base body, further the stent base body, may also include plastic, and may further include polyether urethane, and/or ceramic and/or further polymer coatings.

When endovascularly implantable stents, further coronary stents, are used as implantable base bodies, any customary stent geometries may be used for the present invention. Some stent geometries are described in particular in U.S. Pat. No. 6,896,695, US 2006/241742, U.S. Pat. No. 5,968,083 (Tenax), EP 1 430 854 (helix design), U.S. Pat. No. 6,197,047, and EP 0 884 985.

Within the meaning of the present invention, “coating having one or more aptamers which are the same or different” means that one or more aptamers, which are the same or different, and further may be present either on all or part of the surface of the implant base body, or in a cavity or cavities.

In some embodiments, an implant is implanted in a cavity in the human or animal body, and further in a blood vessel, and on the one hand has a surface in contact with the tissue (abluminal surface) and on the other hand also has a surface in contact with the interior of the cavity, preferably the blood vessel (luminal surface), the coating according to the invention is preferably present on (a portion of) the abluminal surface of the implant according to the invention. By use of this system the risk of adverse side effects, in particular as the result of activation of integrin receptors on cells in the cavity, preferably the bloodstream, for example on platelets, may be reduced.

In one embodiment, an implant is characterized in that the aptamers, independently of one another, (i) can be released or (ii) essentially cannot be released from the coating under physiological conditions.

(i) Release of an Aptamer from the Coating

Within the meaning of the present patent application, (i) “release of an aptamer from the coating” means that the aptamer is suitably coated on the implant base body so that as the result of a local release from the coating the aptamer is able to activate αᵥβ₃ integrin receptors in the local vicinity of the implant, preferably on endothelial cells.

Examples are provided below for appropriate coatings according to some invention embodiments which are suitable for (i) releasing aptamers under physiological conditions:
Coating of all or part of the surface of the implant base body with a polymer-aptamer mixture, wherein the polymer may be of natural or synthetic origin and/or may be degradable or nondegradable.
For example, an implant base body may be coated with a composite containing aptamers and one or more, preferably hydrophobic, biodegradable polymers. Suitable composites are described in DE 10 2006 038 240.4 (BIOTRONIK VI Patent AG), for example, and in this regard the disclosed content is fully incorporated into the present patent application. Accordingly, suitable composites may be produced as follows, for example:
   (i) Preparation of an aqueous solution from polyethylene glycol (PEG) having an average molecular weight in the range of 600 to 30,000 g/mol, preferably 12,000 to 30,000 g/mol,
   (ii) Addition of an aqueous, optionally buffered solution of aptamers, preferably 0.1 to 7 mg/mL aptamer, particularly preferably 5 mg/mL aptamer, to the aqueous solution from step (i), wherein PEG is preferably present in a 1.2- to 100-fold excess in the resulting aqueous solution,
   (iii) Freezing (preferably at −25° C.) the aqueous solution from step (ii) and subsequent freeze drying of the frozen solution (preferably under reduced pressure, for example 3×10⁻⁶ [bar], over a period of preferably 24 hours, to ensure that the water has been completely removed), obtaining a freeze-dried substrate (cotton-like consistency),
   (iv) Preparation of a solution of a hydrophobic biodegradable polymer, preferably polylactic acid, particularly preferably poly-L-lactic acid, in an organic solvent, preferably acetone, chloroform, ethanol, ethyl acetate, acetylacetone, hexylluoroisopropanol, tetrahydrofuran (THF), or dichloromethane, particularly preferably chloroform,
   (v) Addition of the substrate obtained according to step (iii) to the polymer solution from step (iv), and
   (vi) Removal of the organic solvent, preferably under reduced pressure, obtaining the composite.
Coating a cavity or cavities in the implant base body with a suitable aptamer mixture, further a hydrogel-aptamer mixture, in which the aptamer is preferably present in quantities between 2 to 50% by weight, further between 10 to 25% by weight. In one embodiment the aptamers are released from the cavities by swelling and/or diffusion.
Coating all or part of the surface of the implant base body with nanoparticles containing the polymer and aptamer, wherein the polymer may be of natural or synthetic origin and/or may be degradable or nondegradable, and preferably is a positively charged polymer, for example hydrophilic polymers such as polyethyleneimine, polylysine, and chitosan, or hydrophobic polymers such as polylactides, preferably poly(L-lactide), poly(D-lactide), poly(D,L-lactide), and blends thereof, and copolymers, preferably poly(L-lactide-coglycolide), poly(D,L-lactide-coglycolide), poly(L-lactide-co-D,L-lactide), and poly(L-lactide-cotrimethylene carbonate).
The following is an example for producing nanoparticles comprising polyethyleneimine and/or polylysine and aptamers: The positively charged hydrophilic polymer together with the negatively charged nucleic acid of the aptamer in water results in insoluble deposits, which at an appropriate stirring speed (300 rpm, for example) and addition of detergents, for example 0.1% alkylphenylpolyethylene glycol (Triton®X100), precipitate as nanoparticles.
The following is an example for producing nanoparticles comprising chitosan and aptamers: 5 mg chitosan is placed in 1 mL of a 0.5% by weight acetic acid solution. To this solution is added 1 mL aptamer solution (5.5 mg/mL), and vigorous stirring is performed. The fine precipitate is preferably taken up in 50 mM of a phosphate-buffered saline solution (PBS buffer) and further processed.
For the production of nanoparticles comprising hydrophobic polymers and aptamers, emulsion processes (water/oil/water or water/oil/oil) may be used to convert the pure, water-soluble aptamers to a polymer which is insoluble in water. Corresponding production methods may be obtained, for example, from “Biomaterials for Delivery and Targeting of Protein and Nucleic Acids”: ISBN 0-8493-2334-7 (2005) CRC Press; Edited by Ram I. Mahato, which is herein incorporated by reference.
   (ii) Coating is Essentially not Released from the Aptamers

Within the meaning of the present patent application, (ii) “coating is essentially not released from the aptamers” means that the aptamer is bound, directly or via one or more anchor groups which are the same or different, to the optionally functionalized surface of the implant base body in such a way that under physiological conditions the aptamer is essentially not released over an indefinite period of time after implantation. In an appropriate coating the aptamer locally activates αᵥβ₃ integrin receptors in the vicinity of the implantation, preferably on adjacent endothelial cells. This embodiment has an advantage that, due to the absence of polymer in the coating, no polymer degradation products are present, and therefore the risk of correspondingly induced inflammation or thromboses is reduced. Furthermore, the use according to the invention of aptamers which are essentially not released from the coating results in a simplified registration process due to the absence of a substance to be released and a polymer coating.

Suitable anchor groups include, for example, 1,4-phenylene diisothiocyanate, N-succinimidyl-3-(2-pyridyldithio)propionate, and/or a linear or star-shaped PEG block copolymer (also see WO 2004/055153, herein incorporated by reference), or may be selected from the group of compounds of general formula (I) (also see DE 10 2008 040 573.6, BIOTRONIK VI Patent AG, herein incorporated by reference).

(R²O)₂(O)P-L-(CH₂)x-M-R¹ (I)

where
R¹ stands for —COOH, —OH, —SH, —NH₂, benzophenone, or benzophenone derivatives,
R² stands for hydrogen, —CH₂CH₃, or —CH₃,
L stands for a single bond or —O—,
M stands for a single bond or —(CH₂—CH₂—O)y,
x stands for an integer selected from the group comprising 1 to 25, and
y stands for an integer selected from the group comprising 1 to 25.

Within the meaning of the present invention, the term “benzophenone derivative” includes all customary derivatives. Derivatives selected from the group of oxybenzophenones are preferred.

Accordingly, compounds of general formula (I) include, but are not limited to, phosphonic acids of general formula (II)

(HO)₂(O)P—O(CH₂)x-M-R¹ (II)

and

esters thereof of general formula (III)

(R²O₂(O)P—CH₂)x-M-R¹ (III),

and

compounds of phosphorous acid of general formula (IV)

(HO)₂(O)P—O—(CH₂)x-M-R¹ (IV)

and/or esters thereof of general formula (V)

(R²O)₂(O)P—O(CH₂)x-M-R¹ (V),

wherein radicals
R¹, R², M, and x are defined according to general formula (I), and radicals may be present independently of one another.

Compounds of general formula (I) may be further selected from the group comprising hydroxyundecylphosphonic acid, 3-(4-oxybenzophenone)propylphosphonic acid, and carboxydodecylphosphonic acid.

Compounds of general formulas (I), (II), (III), (IV), and (V), wherein M stands for an oligoethylene glycol linker —((CH₂)CH₂O)Y—, are preferred, since this also results in nonspecific protein accumulation at these anchor groups in contrast to anchor groups in which M stands for a single bond.

Methods for immobilizing aptamers are alternatively described in “Immobilisierung von Oligonukleotiden an aminofunktionalisierte Silizium-Wafer” (U. Hacker, Chem. Diss., Hamburg; Use of 1,4-phenylene diisothiocyanate) and in “Miniaturisierte Affinitätsanalytik—Ortsaufgelöste Oberflachenmodifikationen, Assays and Detektion” (I. Stemmler, Chem. Diss., Tübingen, 1999). Methods of coating synthetic surfaces with (cyclic) RGD peptides, including arginine-glycine-asparagine, as integrin ligands (in particular αᵥβ₃ integrin receptors), are described in “Funktionalisierung künstlicher Oberflächen mit Integrinliganden zur Stimulierung integrinvermittelter Zelladhäsion” (Dissertation, Jörg Auernheimer, Munich Technical University, Department of Chemistry, 2005). Methods of coating synthetic surfaces with Implants are described in WO 2004/055153 which are coated with aptamers for achieving adhesion of biological material to the implant. These references are herein incorporated by reference.

According to a further embodiment of the invention, an implant according to the invention may additionally include one or more active substances. Within the meaning of the present invention, an active substance is a substance or a compound which brings about a biological reaction in the human or animal body. In this sense, “active substance” may also be used synonymously with “drug” or “pharmaceutical.” Further, a stent may be coated with one or more active substances in a concentration that is sufficient for bringing about the desired physiological reactions.

Active substances may be selected, for example, from the group comprising: antiphlogistic agents, preferably dexamethasone, methylprednisolone, and diclofenac; cytostatic agents, preferably paclitaxel, colchicine, actinomycin D, and methotrexate; immunosuppressants, preferably limus compounds, particularly preferably sirolimus (rapamycin), zotarolimus (ABT-578), tacrolimus (FK-506), everolimus, biolimus, in particular biolimus A9 and pimecrolimus, cyclosporin A, and mycophenolic acid; platelet aggregation inhibitors, preferably abciximab and iloprost; statins, preferably simvastatin, mevastatin, atorvastatin, lovastatin, pitavastatin, and fluvastatin; and estrogens, preferably 17b-estradiol, daidzein, and genistein; lipid regulators, preferably fibrates; immunosuppressants; vasodilators, preferably satans; calcium channel blockers; calcineurin inhibitors, preferably tacrolimus; anti-inflammatory agents, preferably imidazole; antiallergic agents; oligonucleotides, preferably decoy oligodeoxynucleotide (dODN); endothelium formers, preferably fibrin; steroids; proteins/peptides; proliferation inhibitors; analgesics, and antirheumatic agents.

Further active substance(s) are paclitaxel and limus compounds, preferably sirolimus (rapamycin), zotarolimus (ABT-578), tacrolimus (FK-506), everolimus, biolimus, in particular biolimus A9 and pimecrolimus, preferably rapamycin (sirolimus).

In some embodiments, a peripheral or coronary stent may be coated with the active substance(s) on (a portion of) the abluminal surface, i.e., the surface which after implantation is primarily in contact with the surrounding tissue and not with the vessel lumen of the blood vessel, since for a luminal coating the degradation of the stent, including a biodegradable stent and further including a biodegradable metal stent, may be greatly impaired.

In a further embodiment, an implant which is coated with active substance(s) optionally has one more further coatings on all or a portion of the surface of the implant base body, further on the surface of the layer containing active substance, as a “topcoat” in order to reduce the abrasion on the active substance coating during implantation.

When one or more different polymers are used for the active substance coating and/or the topcoat, the polymers may be selected, for example, from the group comprising:
Nondegradable polymers: polyethylene; polyvinyl chloride; polyacrylates, preferably polyethyl and polymethyl acrylates, polymethyl methacrylate, polymethyl-coethyl acrylate, and ethylene/ethyl acrylate; polytetrafluoroethylene, preferably ethylene/chlorotrifluoroethylene copolymers, ethylene/tetrafluoroethylene copolymers; polyamides (PA), preferably polyamidimide, PA-11, PA-12, PA-46, or PA-66; polyetherimide; polyethersulfone; poly(iso)butylene; polyvinyl chloride; polyvinyl fluoride; polyvinyl alcohol; polyurethane; polybutylene terephthalate; silicones; polyphosphazene; polymer foams, preferably polymer foams of carbonates, styrenes; copolymers and/or blends of the listed polymer classes, polymers of the class of thermoplasts, and
Degradable polymers: polydioxanone; polyglycolide; polycaprolactone; polylactides, preferably poly-L-lactide, poly-D,L-lactide, and copolymers and blends thereof, preferably poly(L-lactide-coglycolide), poly(D,L-lactide-coglycolide), poly(L-lactide-co-D,L-lactide), poly(L-lactide-cotrimethylene carbonate); triblock copolymers; polysaccharides, preferably chitosan, levan, hyaluronic acid, heparin, dextran, cellulose; polyhydroxyvalerate; ethyl vinyl acetate; polyethylene oxide; polyphosphoryl choline; fibrin; albumin; polyhydroxybutyric acid, preferably atactic, isotactic, and/or syndiotactic polyhydroxybutyric acid, and blends thereof.

In some embodiments, polymers for the layer containing active substance and/or the topcoat of the present invention are the degradable polymers described above, since as a result of the complete degradation of the polymer(s) no component foreign to the body remains in the organism.

Embodiments of an implant, and further a stent, may be combined with one another in any conceivable variant, and/or with further embodiments disclosed herein.

According to another embodiment, a process for producing an implant is provided in which the aptamers may be released from the coating independently of one another.

A suitable implant base body which is produced in step a) is described in conjunction with an implant. An implant base body, and further a stent base body, may also be functionalized or derivatized, for example by use of a parylene layer, in particular an aminofunctionalized parylene layer.

An implant base body, and further a stent base body, may optionally be purified using customary methods.

A suitable coating material containing one or more aptamers which are the same or different, having an activating effect on the αᵥβ₃ integrin receptor, wherein the coating material is suitable for releasing the aptamer(s) from the coating under physiological conditions, is likewise described in conjunction with an implant in some embodiments. According to one embodiment, a composite composed of aptamer(s) and a hydrophobic biodegradable polymer is used.

Application of a coating of an implant base body, and further a stent base body, together with the coating material from step b) on at least a portion of the surface or in a cavity or cavities of the implant base body may be carried out using customary methods. In particular, for this purpose the coating material from step b) may be applied to the surface of the implant base body using a dipping process (dip coating), spray coating using a single- or multicomponent nozzle, rotary atomization, and pressurized nozzles or sputtering. A cavity or cavities may typically be selectively coated by brushing or filling.

When an implant additionally includes one or more active substances, these may either be contained in the coating material from step b), or the active substance(s) may be applied in a separate layer on all or a portion of the surface of the implant base body, and further on a surface of the implant base body which is not coated with aptamer, using customary methods. For this purpose, a pure active substance melt, an active substance-solvent mixture, or an active substance-polymer mixture may be applied to all or a portion of the surface of the implant base body using, for example, a dipping process (dip coating), spray coating using a single- or multicomponent nozzle, rotary atomization, and pressurized nozzles or sputtering. When one or more topcoats are also present on the implant, the above coating methods, for example, may also be used for this purpose.

When primarily the abluminal surface of a stent is to be coated with aptamers, one or more active substances, and/or a topcoat, this may also be carried out by the fact that in the above-mentioned processes an implant base body, and further a stent base body, is placed on a cylinder, cannula, or mandrel, for example, so that only the abluminal surface of the stent is coated with an aptamer or an active substance layer. Alternatively, the abluminal coating may be carried out by roller application, or via selective application by brushing or filling of cavities.

One or more coating steps may optionally be followed by a customary drying step or other common physical or chemical postprocessing steps, for example vacuum or plasma treatment, before an implant, and further a stent, is further treated.

According to another embodiment, a process is provided for producing an implant in which the aptamer (ii) is essentially not released from the coating.

A suitable implant base body which may be provided in step a) is also described here in conjunction with the implant. In one embodiment, this implant is neither functionalized nor derivatized.

In a further embodiment, suitable anchor groups which are provided in step b) are likewise described in conjunction with an implant wherein these anchor groups are usually present dissolved, suspended, or emulsified in a solvent preferably selected from the group comprising methanol, ethanol, acetone, tetrahydrofuran (THF), dimethylformamide (DMF), chloroform, dimethylsulfoxide (DMSO), and dichloromethane.

In one embodiment of the invention, an implant base body, and further a stent base body, provided in step a) is purified using customary methods so that the base body is activated in such a way that the anchor groups provided in step b) may be bound in step c). An implant base body, and further a stent base body, may be purified in an oxygen plasma and/or by rinsing with solvent, preferably the solvent series dichloromethane, acetone, methanol, and Millipore water. This purification step may optionally be followed by a customary drying step.

Functionalization according to an invention embodiment of the implant base body, and further a stent base body, in step c) with the anchor group(s) from step b) usually refers to the complete or partial bringing of the (purified) implant base body, and further a stent base body, into contact with one or more solutions of the same or different anchor groups, and the solvent(s) are then completely or partially removed, preferably evaporated. In one embodiment, the solvent(s) are removed, preferably evaporated, within a period of one hour in such a way that the meniscus of the solution migrates over the implant surface, further a stent surface. According to the invention, “bringing into contact” typically means spraying of the purified base body with, or dipping the purified base body in, one or more solutions of the same or different anchor groups.

An implant base body, and further a stent base body, which is functionalized with one or more anchor groups may then optionally be thermally treated (tempered) to increase the binding of the anchor groups to the base body. Tempering may be, for example, carried out over a period of 1 to 124 hours and/or in a temperature range of 60° C. to 120° C., preferably for 18 to 74 hours at 100° C. to 140° C.

The functionalized implant base body, and further the stent base body (functionalized or not), may then be rinsed with solvent.

A functionalized implant base body, and further a stent base body, pretreated in this manner may then be optionally partially or completely placed in a carbonyl diimidazole (CDI) solution, preferably in dry dioxane. The base body may be placed in a 0.3 M solution of carbonyl diimidazole in dry dioxane for 15 hours. This is optionally followed by rinsing with dry dioxane and/or a drying step.

In some embodiments, suitable aptamers which are provided in step d) are likewise described in conjunction with an implant, and are usually provided in the form of a solution, suspension, or emulsion. In one embodiment the aptamers are present in a buffer solution that may be free of amino acids and/or RNase, preferably PBS buffer (phosphate-buffered saline), MES buffer (2-morpholinoethanesulfonic acid), borate buffer, etc., preferably PBS buffer.

In some embodiments, in step e) the implant base body, and further a stent base body, from step c) and functionalized is completely or partially brought into contact with the solution, suspension, or emulsion of the aptamers from step d) and optionally rinsed. “Bringing into contact” is typically understood to mean spraying of the functionalized base body with, or dipping in, solutions, suspensions, or emulsions of the same or different aptamers. In one embodiment, this is followed by a drying step, preferably using nitrogen.

When compounds of formula (I), (II), (III), or (IV) having benzophenone anchor groups are used, for forming the bond of aptamers to the implant base body, and further a stent base body, this may be followed by a light exposure step, typically above 260 nm, preferably at 360 nm, at 100 mW/cm².

In some embodiments, for formation of the bond of the endothelial cells to carbonyl anchor groups of compounds of formula (I), (II), (III), or (IV), this may be followed first by activation of the carbonyl anchor group using N,N-(3-dimethylaminopropyl)-N′-ethylcarbodiimide hydrochloride (EDC) and/or N-hydroxysuccinimide (NHS), and only then, coupling with the aptamer to be used according to the invention.

For the production process provided, embodiments of the features, also described in conjunction with the first embodiment of the implant, may be combined with one another, provided that such is meaningful for one skilled in the art.

For another embodiment of the use of an aptamer having an activating effect on the αᵥβ₃ integrin receptor for mediating endothelial cell adhesion to an implant for a human or animal body, embodiments and features also described in conjunction with the first, second, or third embodiment of the implant and the production process therefor may likewise be combined with one another, provided that such is meaningful for one skilled in the art.

For another embodiment of the use of one or more different aptamers having an activating effect on the αᵥβ₃ integrin receptor for the complete or partial coating of an implant base body for a human or animal body, embodiments and features also described in conjunction with the first, second, or third embodiment of an implant and a production process therefor may likewise be combined with one another, provided that such is meaningful for one skilled in the art.

For another embodiment of the use of one or more different aptamers having an activating effect on the αᵥβ₃ integrin receptor for producing an implant, embodiments and features also described in conjunction with the first, second, or third embodiment of the implant and the production process therefor may likewise be combined with one another, provided that such is meaningful for one skilled in the art.

### EXEMPLARY EMBODIMENTS

Some further aspects of the present invention are described below by use of exemplary embodiments which, however, do not limit the scope of protection of the subject matter according to the invention.

### Example 1

### Aptamer Coating for a Stent Base Body from which Aptamers May be Released

### Production of the Aptamer Coating Material:

Polyethylene glycol (PEG) having a molar mass of 12,000-30,000 g/mol was dissolved at room temperature in water, preferably distilled water. To this solution was added, likewise at room temperature, an aqueous or buffered solution of one or more aptamers (5 mg/mL) having an activating effect on the αᵥβ₃ integrin receptor, so that the PEG was present in a 1.2- to 100-fold gravimetric excess. After preparation of a homogeneous solution, the solution thus obtained was cooled to −25°, the resulting frozen substance was mechanically crushed, and freeze-drying was then performed at reduced pressure (3×10⁻⁶ bar), over a period of 24 hours, to ensure that water was essentially completely removed.

A cotton-like substance was obtained. This cotton-like substance was soluble in chloroform, and was added to a hydrophobic biodegradable polymer, preferably a polylactide, preferably poly(L-lactide), poly(D,L-lactide), and copolymers and blends thereof, preferably poly(L-lactide-coglycolide), poly(D,L-lactide-coglycolide), poly(L-lactide-co-D,L-lactide), poly(L-lactide-cotrimethylene carbonate), etc., dissolved in chloroform. The aptamer-PEG complex was present in solubilized (quasi-dissolved) form.

The surface of a stent base body optionally purified in oxygen plasma (instrument from Diener; Tetra model; 200 W; 0.2 mbar; 2-30 min) or by rinsing with the solvent series dichloromethane, acetone, methanol, and Millipore water was completely or partially coated with the aptamer coating material produced as described above.

### Example 2

### Aptamer Coating for a Stent Base Body from which Aptamer Essentially Cannot be Released

A stent purified in oxygen plasma (instrument from Diener; Tetra model; 200 W; 0.2 mbar; 2-30 mM) or by rinsing with the solvent series dichloromethane, acetone, methanol, and Millipore water was further treated as follows:

A 1 mM solution of hydroxyundecylphosphonic acid in dry tetrahydrofuran was prepared. The stent was suspended therein, and the solvent was evaporated over a period of one hour, whereby the meniscus of the solution migrated over the stent surface.

The stent was then tempered for 18 h at 120° C. and then rinsed in the solvent. The stent pretreated in this manner was placed in a 0.3 M solution of carbonyl diimidazole (CDI) in dry dioxane for 15 h. The stent was then rinsed 2×10 mM with dry dioxane and dried in a nitrogen stream.

A solution of the above-described aptamers to be used according to the invention (approximately 50 μg/mL) in PBS buffer (preferably free of amino acids and RNase) was added to the stents treated in this manner, and was agitated overnight at 4° C. The stents were then rinsed with buffer.

### Example 3

### Aptamer Coating for a Stent Base Body from which Aptamer Essentially Cannot be Released

A stent purified in oxygen plasma (instrument from Diener; Tetra model; 200 W; 0.2 mbar; 2-30 min) or by rinsing with the solvent series dichloromethane, acetone, methanol, and Millipore water was further treated as follows:

A 3 mM solution of 3-(4-oxybenzophenone) propylphosphonic acid in dry tetrahydrofuran was prepared. The stent was sprayed 3× with this solution.

The stent was then tempered for 12 h at 120° C., and then rinsed in the solvent.

These stents were added to a solution of the above-described aptamers to be used according to the invention (approximately 50 μg/mL) in PBS buffer (free of amino acids and RNase) and agitated overnight at 4° C.

On the next day the stents were removed from the solvent, dried, and exposed to light at 360 nm at 100 mW/cm². Non-bound RNA was washed off.

### Example 4

### Aptamer Coating for a Stent Base Body from which Aptamer Essentially Cannot be Released

A stent purified in oxygen plasma (instrument from Diener; Tetra model; 200 W; 0.2 mbar; 2-30 min) or by rinsing with the solvent series dichloromethane, acetone, methanol, and Millipore water was further treated as follows:

A 1 mM solution of carboxydodecylphosphonic acid in dry tetrahydrofuran was prepared. The stent was suspended therein, and the solvent was evaporated over a period of one hour, whereby the meniscus of the solution migrated over the stent surface. The stent was tempered for 74 h at 120° C., and was then rinsed in the solvent. The stent pretreated in this manner was placed in a 1:1 mixture of 0.4 M EDC and 0.1 M NHS in Millipore water for 45 min. The stent was then rinsed briefly with Millipore water and dried in a nitrogen stream.

A solution of the above-described aptamers to be used according to the invention (approximately 50 μg/mL) in PBS buffer (free of amino acids and RNase) was added to the stents treated in this manner, and was agitated overnight at 4° C. The stents were then rinsed with buffer.

What is claimed is:

1. An implant for a human or animal body comprising a base body having a coating, comprising at least one aptimer that activates an αᵥβ₃ integrin receptor.

2. An implant according to claim 1, wherein the implant base body is selected from the group consisting of cardiac pacemakers, cardiac implants, pacemaker electrodes, defibrillation electrodes, shunts, biodegradable or permanent, coronary stents, peripheral stents, and neurostents.

3. An implant according to claim 1, wherein the aptamer(s) are composed of nucleic acids of aptamers which bind to the αᵥβ₃ integrin receptor but inhibit endothelial cell adhesion, and one or more phosphate groups of the sugar-phosphate backbone are derivatized.

4. An implant according to claim 3, wherein the nucleic acid aptamer is SEQ ID NO:1.

5. An implant according to claim 3, wherein the derivations are chosen from thiophosphate and methyl phosphate groups.

6. An implant according to claim 1, wherein the coating is present on at least part of the surface of the base body.

7. An implant according to claim 1, wherein the coating is present in at least one cavity in the implant base body.

8. An implant according to claim 1, wherein the coating is present on the abluminal side of the implant.

9. An implant according to claim 1, wherein at least one aptamer is released from the coating under physiological conditions.

10. An implant according to claim 1, wherein at least one aptimer essentially cannot be released from the coating under physiological conditions.

11. An implant according to claim 1, wherein the at least one aptamer are present in a coating which comprises hydrophobic biodegradable polymers.

12. An implant according to claim 1 wherein the at least one aptamer(s) essentially cannot be released from the coating and are bound independently of one another via one or more anchor groups to the surface of the base body

13. An implant according to claim 1, wherein the implant also contains at least one active substance selected from the group comprising: antiphlogistic agents, cytostatic agents, immunosuppressants, platelet aggregation inhibitors, statins, estrogens, lipid regulators, immunosuppressants, vasodilators, calcium channel blockers, calcineurin inhibitors, anti-inflammatory agents, antiallergic agents, oligonucleotides, endothelium formers, steroids, proteins/peptides, proliferation inhibitors, analgesics, and antirheumatic agents.

14. An implant according to claim 13 wherein:
the antiphlogistic agents are selected from the group consisting of dexamethasone, methylprednisolone, and diclofenac;
the cytostatic agents are selected from the group consisting of paclitaxel, colchicine, actinomycin D, and methotrexate;
the immunosuppressants are selected from the group consisting of limus compounds, sirolimus (rapamycin), zotarolimus (ABT-578), tacrolimus (FK-506), everolimus, biolimus, biolimus A9, pimecrolimus, cyclosporin A, and mycophenolic acid;
the platelet aggregation inhibitors are selected from the group consisting of abciximab and iloprost;
the statins are selected from the group consisting of simvastatin, mevastatin, atorvastatin, lovastatin, pitavastatin, fluvastatin, estrogens, 17b-estradiol, daidzein, genistein;
the lipid regulators are fibrates;
the vasodilators are satans;
the calcineurin inhibitors are tacrolimus;
the anti-inflammatory agents are imidazole;
the oligonucleotides are decoy oligodeoxynucleotide (dODN); and,
the endothelium formers are fibrin.

15. An implant according to claim 1 made by a process comprising the following steps:
a) providing the implant base body,
b) providing the coating material comprising at least one aptamer which has an activating effect on the αᵥβ₃ integrin receptor, the coating material being suitable for releasing the aptamer(s) from the coating under physiological conditions, and
c) applying the coating material from step b) to at least a portion of the implant base body.

16. An implant according to claim 1 made by a process comprising the following steps:
a) providing the implant base body,
b) providing at least one anchor group,
c) functionalizing the implant base body of step a) using the anchor group(s) of step b),
d) providing at least one aptamer having an activating effect on the αᵥβ₃ integrin receptor, and
e) binding the aptamer(s) from step d) to the functionalized implant base body of step c).

17. A method of treating an implant for a human or animal body comprising the steps of applying at least one aptamer that activates an αᵥβ₃ integrin receptor for mediating endothelial cell adhesion to the implant.

18. A method of treating an implant for a human or animal body comprising the steps of coating an implant base body with at least one aptamer having an activating effect on the αᵥβ₃ integrin receptor.

19. An implant according to claim 1 made by using one or more different aptamers having an activating effect on the αᵥβ₃ integrin receptor.

20. An implant for a human or animal body comprising:
a base body having a surface;
a coating covering at least a portion of the surface, the coating comprising at least one aptimer that is SEQ ID No: 1.


SEQUENCE LISTING (1 sequence)

SEQ ID NO: 1 - DNA, 80 bp, Artificial Sequence
  Sequence binds alpha v beta 3 integrin receptor
catgcgccttccccctgtggttggtgtcagtcggcctgtgtgattagtggaatgtgcgatgggtctcccagcgggattgg